(19) United States
(12) Patent Application Publication
(10) Pub. No.: US 2018/0045734 A1
(43) Pub. Date: Feb. 15, 2018

(54) Chemically functionalized array to analyze protein modifications

(71) Applicant: TYMORA ANALYTICAL OPERATIONS LLC, West Lafayette, IN (US)

(72) Inventors: Weiguo Andy Tao, West Lafayette, IN (US); Anton B. Ilyuk, West Lafayette, IN (US)

(21) Appl. No.: 15/555,983

(22) PCT Filed: Mar. 6, 2016
(86) PCT No.: PCT/US16/21106

§ 371 Date: Sep. 6, 2017

Related U.S. Application Data
(60) Provisional application No. 62/129,724, filed on Mar. 6, 2015.

(51) Int. Cl. G01N 33/68

(52) U.S. Cl. CPC G01N 33/6842

(57) ABSTRACT

Methods and chemicals for the capture and analysis of a selected group of protein modifications. Post-translational modifications alter the functional groups in proteins. The resulting modified proteome is useful for biomarker discovery, clinical diagnostics, and protein dynamics. Present immunoassays to quantify modified proteins are limited by their dependence on antibodies, which are often not specific for post-translational modifications. This invention utilizes a reagent and platform with a binding moiety that is capable of selectively binding to the modified protein.

### CROSS-REFERENCE TO RELATED APPLICATIONS

This application is a U.S. national phase patent application which claims the benefit of a PCT international parent application PCT/US16/21106, filed Mar. 6, 2016 which claims the benefit of U.S. provisional patent application Ser. No. 62/129,724, filed Mar. 6, 2015, the disclosure of both are expressly incorporated by reference.

### FIELD

This disclosure is directed to materials and methods for the capture and analysis of a selected group of modified proteins.

### BACKGROUND

Disease biomarker discovery and validation is a promising task. Disease biomarker discovery and validation is promising for proteomics platforms, such as biomarkers of post-translational modifications (PTMs). PTMs, such as phosphorylation, oxidation, methylation, and glycosylation, are major players in cancer formation, cancer progression, and other disease progression. Proteins with PTMs are primary targets for multiple therapeutics.

Immunoassays detect and quantify biological molecules (i.e. proteins) by using antibodies that bind to the molecule. The antibody may be labeled (i.e. enzyme, radioactive isotopes, fluorophores, or electrochemiluminescent tags) to facilitate detection of the complex after binding. Enzyme-linked immunosorbent assays (ELISAs) label the detection antibody with an enzyme (i.e. horseradish peroxidase) that produces an observable color change when combined with the enzyme's substrate. Sometimes the enzyme is linked to a second detection antibody to avoid the expense of creating a primary detection antibody specific to the antigen. In ELISA, a sample is applied to a surface for analysis and then probed with the reporter antibody(s). The sample can bind non-specifically to the surface via adsorption, or specifically using another capture antibody. Thus, the most specific immunoassays “sandwich” the antigen between a capture antibody and the detection antibody(s).

Multiple studies have been carried out to examine the quality of available antibodies. The consensus arising from these is that less than 20% of antibodies are of sufficient quality (See FIG. 5 of Haab, B. B., Dunham, M. J., and Brown, P. O. (2001) Protein microarrays for highly parallel detection and quantitation of specific proteins and antibodies in complex solutions. Genome biology 2, RESEARCH0004.). Less than 5% of antibodies exhibit sufficient on-chip performance or can be used for antibody pair matching. Many antibodies may display sufficient monospecific function in the initial testing, but in combination with multiple antibodies and antigens exhibit significant cross-reactivity from non-matched pairs. To alleviate some of these drawbacks, reverse-phase protein arrays have been developed.

Reverse phase protein array (RPPA) is a descendent of the classic immunoassays that sandwich the antigen between antibodies. RPPA analysis is an efficient multiplexed immunoassay approach with quantitative capabilities. RPPA entails direct spotting of the sample of interest onto a high-binding platform to adsorb the complete proteome of the sample in a very small area. The presence of a protein of interest can be then detected using its validated antibody. In other words, in the “reverse phase” the antigen is bound to a solid phase, usually nitrocellulose, and subsequently probed with an antibody or other affinity reagent.

RPPA permits quantitative analysis of the antigen of interest such as a modified protein such as phosphorylated, glycosylated, acetylated, cleaved or total cellular proteins. The approach allows direct semi-quantitative comparison among multiple samples for the same target, as long as they can be printed on the same platform. This process is also beneficial because a relatively small amount of starting material is needed, making RPPA a beneficial choice for clinical samples like needle biopsies, biological fluids or microdissected tissues.

RPPA can be used for protein signal pathway mapping in animal models, in cell lines and xenografts, and in clinical sample profiling. Biological samples can include enriched cell populations from tissue microdissection, from direct extraction of heterogeneous tissue samples, cell lines or subcellular fractions, or serum or plasma. RPPA is an available method that can quantitatively measure large numbers of low abundance analytes in biological samples. RPPA is used for proteomic profiling, diagnostic marker identification, protein kinetic monitoring, and for experimental validation of theoretical models.

There have been multiple studies describing the use of emerging reverse phase arrays for clinical sample analysis, including for leukemia, breast cancer, prostate cancer studies, thyroid cancer, lung cancer and many others. For example, see Irwin, M. E., Nelson, L. D., Santiago-O'Farrill, J. M., Knouse, P. D., Miller, C. P., Palla, S. L., Siwak, D. R., Mills, G. B., Estrov, Z., Li, S., Kornblau, S. M., Hughes, D. P., and Chandra, J. (2013) Small molecule ErbB inhibitors decrease proliferative signaling and promote apoptosis in philadelphia chromosome-positive acute lymphoblastic leukemia. PLoS One 8, e70608; Gollapudi, K., Galet, C., Grogan, T., Zhang, H., Said, J. W., Huang, J., Elashoff, D., Freedland, S. J., Rettig, M., and Aronson, W. J. (2013) Association between tumor-associated macrophage infiltration, high grade prostate cancer, and biochemical recurrence after radical prostatectomy. American journal of cancer research 3, 523-529; Cheng, S., Serra, S., Mercado, M., Ezzat, S., and Asa, S. L. (2011) A high-throughput proteomic approach provides distinct signatures for thyroid cancer behavior. Clinical cancer research: an official journal of the American Association for Cancer Research 17, 2385-2394; and Nanjundan, M., Byers, L. A., Carey, M. S., Siwak, D. R., Raso, M. G., Diao, L., Wang, J., Coombes, K. R., Roth, J. A., Mills, G. B., Wistuba, I I, Minna, J. D., and Heymach, J. V. (2010) Proteomic profiling identifies pathways dysregulated in non-small cell lung cancer and an inverse association of AMPK and adhesion pathways with recurrence. Journal of thoracic oncology: official publication of the International Association for the Study of Lung Cancer 5, 1894-1904.

RPPA is viewed as a beneficial method due to a need for a relatively small amount of material, sample-to-sample comparison capabilities, and often sub-picogram sensitivity. However, arguably the most notable advantage of RPPAs is the requirement of only one validated detection antibody per target. RPPA eliminates the need for capture antibodies and antibody pairs. Eliminating the need for capture antibodies and antibody pairs in general has therefore advanced RPPAs as a viable option for large scale protein analyses, especially for clinical samples.

RPPA is dependent on the availability of high quality monospecific affinity antibodies, antibodies that can detect an antigen, such as a protein or a modified protein, with high affinity and specificity. In order to validate an antibody, it must be shown to be specific, selective, and reproducible in the method's sample matrix. The antibody must perform robustly across different sample types and over time.

Multiple studies have been carried out to examine the quality of available antibodies. Many antibodies may display sufficient monospecific function in the initial testing, but in combination with multiple antibodies and antigens many antibodies exhibit cross-reactivity from non-matched pairs. Additionally, many antibodies are made against peptides and require antigen denaturation. But denaturation destroys protein-protein interactions and may reduce functional groups in particular modifications.

RPPAs only enable the detection of proteins or modified proteins with available validated antibodies. High-quality antibodies are currently available for only a small percentage of the known proteins involved in signal networks and gene regulation. There is a lack of antibodies specific for the modification or activation state of the target protein. Detection of modified proteins by modification-specific antibodies has been limited in RPPA assays, primarily due to the lower relative quality of modification-specific antibodies compared to antibodies for total protein. There are also a significant number of modified sites present, most likely hundreds of thousands, with most not having any commercial antibodies. Moreover, the number of different types of proteins in an enriched mixture increases the complexity to detect low abundant modified proteins (e.g. phosphoroproteins).

Current limitations for RPPA of PTM analysis include restricted availability and generally low specificity of site-specific antibodies, low throughput, and the lack of quantitative detection. Despite frequent usage of the currently available phospho- and other PTM-specific antibodies, there are multiple shortcomings that limit their applications. First, in order to use the assay, the site of modification of interest has to be known beforehand, thus limiting the analysis to only well-characterized signaling events. Second, an effective site-specific antibody has to be made for every single modified residue and protein, making the assays rather costly and difficult if the antibody is unavailable. Third and finally, while much progress has been made in the field of antibody generation, development of PTM-specific antibodies faces the challenges of poor selectivity, overall reduced quality, and high cost.

Despite the shortcomings, antibody-based detection of PTMs remains the primary mode of analysis. To enable more large-scale examination of protein expression levels and regulation mechanisms, antibody microarray technology has been developed and heavily utilized. Antibody arrays entail immobilization of dozens or hundreds of antibodies raised against relevant proteins in parallel on a single microarray, thus enabling capture of these proteins from a complex mixture. Following this, a form of detection of the captured molecules is required. Most often, for modifications, a PTM-specific antibody is used for a particular site of interest that has been carefully matched with the capture antibody. While useful in many cases, this procedure has a number of disadvantages, including difficulties in developing a good antibody match and detection of only a single modification site.

Currently, reverse-phase arrays only enable the detection of proteins or modifications with available validated antibodies (currently a small fraction of all antibodies). Functional studies utilizing such arrays have been limited to a few select protein domains or binding affinities, such as ankyrin repeat proteins, small molecule or peptide-binding proteins, glycan interactomes and lectin binding (See Blixt, O., and Westerlind, U. (2014) Arraying the post-translational glycoproteome (PTG). Curr Opin Chem Biol 18, 62-69 and Hirabayashi, J., Yamada, M., Kuno, A., and Tateno, H. (2013) Lectin microarrays: concept, principle and applications. Chemical Society reviews 42, 4443-4458.), SH2 binding domains and ligand-binding screening. These function studies are based on affinity interactions using antibodies, proteins and their domains, and other protein-derived affinity molecules.

In light of the limitations of immunoassays and RPPAs there remains a need for high-throughput technologies to analyze modified proteins such as PTM modified proteins. Examination of PTM modified proteins and their dynamics is beneficial to understanding onset and progress of diseases on the molecular level. PTM modified proteins examination allow for comparison of temporal cellular activity, differentiation within cell types, dynamic feedback mechanisms, network crosstalk, modifications during disease formation and progression, and response of a biological system to drug treatments.

Specifically there is also a need for a systems based approach to analyzing a specific type of modified protein, such as all phosphorylated proteins.

### SUMMARY

A composition for binding a modified protein comprised of a pre-assembled binding moiety with an active binding molecule including at least one component selected from the group consisting of a polymer, nanopolymer, a dendrimer molecule, and a spacer link, where the binding moiety is selected from the group consisting of metal ions, hydrazide, hydroxylamine, aldehyde, carbonyl groups, azide, alkyne, thiol, equivalents and combinations thereof, and the binding moiety is also immobilized to a solid support.

A method for the detection of modified biological analyte in a sample comprising the steps of contacting an immobilized binding moiety, which includes an active binding molecule and at least one component selected from the group consisting of a polymer, nanopolymer, a dendrimer molecule, and a spacer link, with a sample, such that the binding moiety selectively binds to a modification of a protein of the sample, removing at least a portion of any unbound sample, and analyzing the protein using a detection moiety selected from the group consisting of antibodies, aptamers, affirmers, equivalents and combinations thereof.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 is a schematic comparing a standard RPPA method with an embodiment of the method disclosed herein.

FIG. 1A illustrates a standard RPPA procedure.

FIG. 1B illustrates a proposed RPPA platform in which the array is first functionalized with modified PolyMAC.

FIG. 2 is a schematic illustrating a procedure of coating an RPPA solid phase, glass, paper, or chip with active binding molecules for capture and analysis of a selected group of modified proteins (i.e. phosphorylation, glycosylation, nitrosylation). The (non)covalently coated solid phase is then exposed to the sample, washed, and subsequently detected with a tagged antibody.

FIG. 3 illustrates a single array where the right half of the array is modified to bind PTM proteins using an embodiment of the method disclosed herein.

FIG. 4 is an illustration showing a comparison between control and titanium-modified membranes for capture of the proteome/phosphoproteome from cell culture. Control or phosphorylated cJun was spiked into lysate at 1:200 ratio, spotted onto both membranes in serial dilutions and detected.

FIG. 5 is an illustration showing samples spotted by a robotic spotting microarrayer. A serial dilution of control cJun is spotted in top half and a serial dilution of phospho cJun is spotted in the bottom half. Control or phosphorylated cJun was spiked into lysate at 1:200 ratio, spotted onto the membrane in serial dilutions and detected.

FIG. 6 is an illustration showing a comparison between control and phospho-cJun capture on the Ti-modified membrane in plasma background. Each spotted sample contained 1 ng cJun and 10 ug plasma protein.

FIG. 7A is a legend to identify regions in the corresponding top half of the array of FIG. 7C.

FIG. 7B is a legend to identify regions in the corresponding bottom half of the array of FIG. 7C.

FIG. 7C shows the detection of AGP on the membrane. PolyMAC-O—NH₂ is the modified coating on the membrane in the top half of the array in FIG. 9C, and the non-coated membrane is in the bottom half of the array in FIG. 9C. Both control and oxidized AGP are spotted on each half in a serial dilution ranging from 125 pg to 200 fg from left to right.

FIG. 8 is a plot of the signal from the serial dilution of the oxidized AGP on the PolyMAC-O—NH₂ coated membrane illustrated in FIG. 7.

FIG. 9A is a legend to identify regions in the corresponding top half of the array of FIG. 9C.

FIG. 9B is a legend to identify regions in the corresponding bottom half of the array of FIG. 9C.

FIG. 9C shows the detection of AGP on the membrane. PolyMAC-O—NH₂ is the modified coating on the membrane in the top half of the array, and the non-coated membrane is in the bottom half. Endogenous AGP protein from plasma directly is spotted on the right side of array and compared to control AGP standard spotted on the left side of array.

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Active binding molecules capable of specifically capturing modified protein groups are described in more detail in U.S. Pat. No. 8,501,486 to Tao and U. S. Published Patent Application 2013/0095502 to Tao et. al., the subject matter of each are expressly incorporated by reference. U.S. Pat. No. 8,501,486 to Tao details a polymer-based metal ion or metal oxide capturing reagent suitable for the capture of phosphopeptides from mixtures (PolyMAC). U. S. Published Patent Application 2013/0095502 to Tao et al. describes a reagent for the detection of phosphorylated molecules.

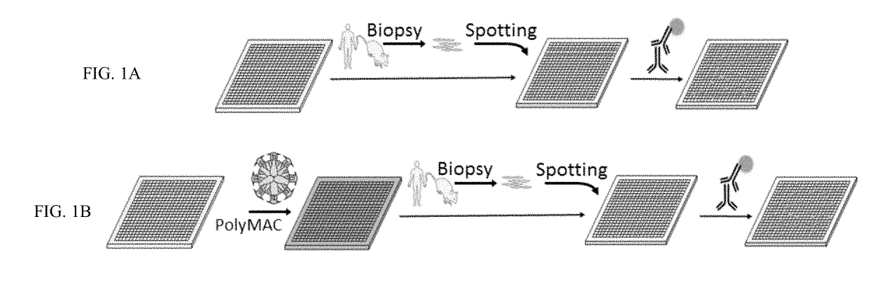

This disclosure compares standard RPPA methods to methods according to embodiments of the current disclosure by immobilizing a binding moiety, such as PolyMAC, to the membrane before spotting the sample. FIG. 1 compares the overall methodology of standard RPPA and functionalized PTM-RPPA (PolyMAC based capture using Ti, O—NH₂ or other chemistries).

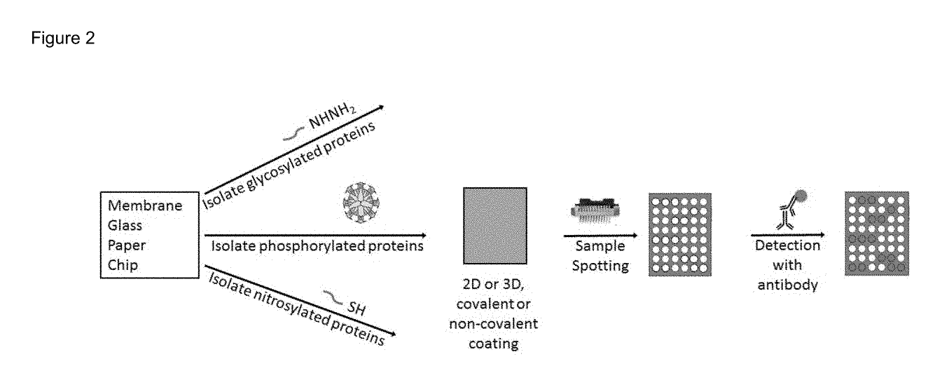

As illustrated in FIG. 2, this disclosure illustrates active binding molecules capable of specifically capturing modified protein groups such as proteins modified by phosphorylation, glycosylation, nitrosylation, sulfonation, oxidation, and the like.

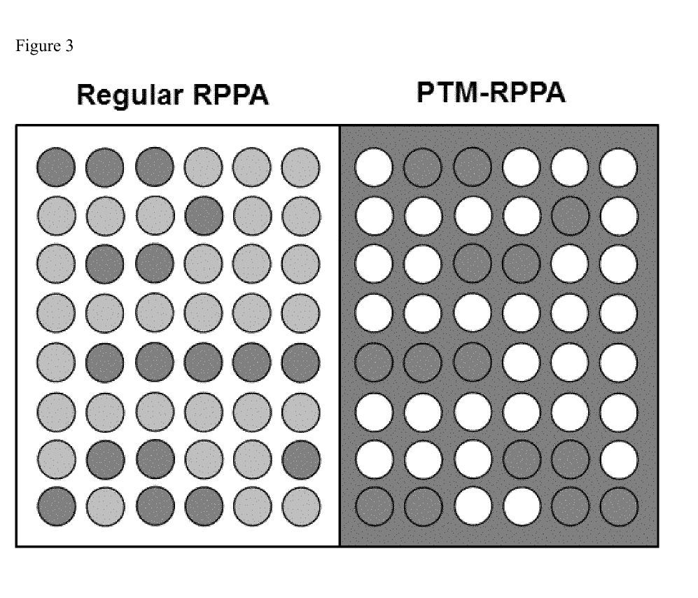

In one example embodiment, overall signal (standard RPPA) and PTM (PTM-RPPA) signal can be detected simultaneously if only half of the array is modified by various PolyMAC reagents. FIG. 3 is a schematic demonstrating how overall signal (standard RPPA) and PTM (PTM-RPPA) can be detected simultaneously if only half of the array is modified by various PolyMAC reagents. Then the same samples can be spotted on both halves, and the target signal detected by the same antibody together, providing two or more distinct data sets.

TABLE 1
Additional binding molecules

| Protein modification | Active chemical moiety |
|---|---|
| Phosphorylation | Metal ions |
| Glycosylation | Hydrazide, hydroxylamine and the like |
| | to capture oxidized sugar moiety |
| Nitrosylation, sulfenylation, | SH or other nucleophilic moiety after |
| sulfhydrylation | blocking of unmodified Cysteins and |
| | removal of modification (pyridyldithiol, |
| | maleimide, thiol and the like) |
| O-glycosylation | Thiol group to achieve Michael addition |
| | after beta-elimination |
| Azide or alkyne incorporation | Alkyne or azide conjugation |
| into proteins or modifying | |
| molecules genetically or | |
| metabolically | |

Additional binding molecules for individual PTMs are summarized in Table 1. Hydrazide and hydroxylamine are example binding molecules to capture the oxidized sugar moiety in glycosylation modifications. Thiol or other nucleophilic moiety may be used as the binding molecule to capture nitrosylations, sulfenylation, and/or sulfhydroxylation modifications.

The active portion of the immobilized active binding and/or capture(coated) molecules can include, but is not limited to, metal ions, hydrazide, hydroxylamine, aldehyde, carbonyl groups, azide, alkyne, thiol and equivalents.

The disclosure may utilize a platform such as an array, glass slide, and/or membrane. The platform is coated, functionalized, and/or immobilized with the active binding molecules. The active binding and/or capture molecules can be immobilized on a platform as is (as small molecules or with a linker) or can be functionalized on the surface of polymers, nanopolymers, dendrimers, gels, beads and the like.

The binding and/or capture function is proposed to not be based on antibodies, aptamers, affimers, lectins or other proteins and nucleic acids. The binding and/or capture function between the active binding and/or capture molecules and analytes is proposed to be chemical in nature, including covalent bonds, ionic bonds, metal chelation, intermolecular bonds and the like. The chemical binding/chelation would enable the capture of the whole or part of a modified proteome onto the platform.

The captured modified proteins can then be detected using the validated antibodies, aptamers, affimers, lectins or any other detection methods as in standard RPPA, with the changes in signal attributable to changes in target modification as illustrated in FIG. 1. In parallel, a standard RPPA-like assay can be carried out to examine changes in protein amount, and compare these to changes in protein modifications.

### Example #1

An example of a phospho-RPPA protocol according to an embodiment of the present disclosure is to: (1) coat nitrocellulose on a glass slide with modified PolyMAC-Ti, let it dry for 1 hr, and then coat again; (2) Incubate with 1% trifluoroacetic acid for 15 min and dry completely; (3) Denature the samples in 2% sodium dodecyl sulfate (SDS) with 20 mM dithiothreitol by boiling for 5 min at 95 degrees Celsius; (4) Make at least 4 serial dilutions of each sample; (5) Spot samples using a pin or a pipette; (6) Wash the array three times for 10 min each wash using a 0.5% SDS in a tris-buffered saline and Tween 20 mixture (TBST), the TBST includes 50 mM Tris(hydroxymethyl)aminomethane, 150 mM NaCl, and 0.05% Polysorbate 20 (aka Tween 20), and then a fourth wash with TBST; (7) Block the array for 1 hr with 1% bovine serum albumin (BSA) in TBST; (8) Incubate the array with the appropriate primary antibody using optimal dilution in 1% BSA in TBST; (9) Wash four times with TBST, for 5 min each wash; (10) Incubate the array with the appropriate secondary antibody using optimal dilution in 1% BSA in TBST; (11) Wash four times with TBST for 5 min each wash; (12) Detect the signal using the method of choice, depending on the secondary antibody conjugate.

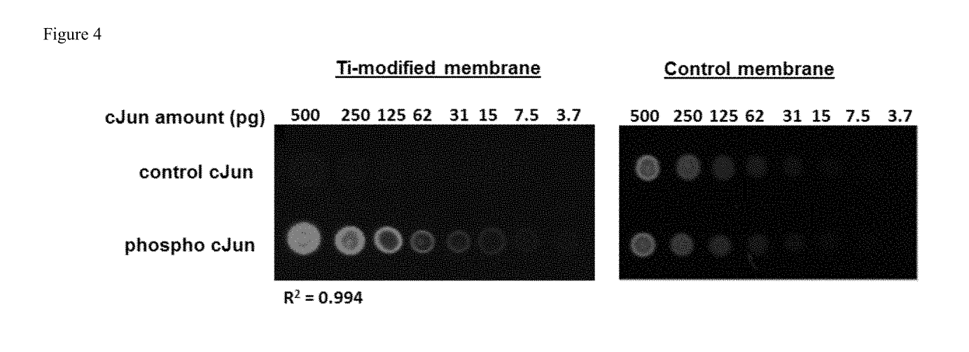

In one example illustrated in FIG. 4, immobilized nitrocellulose membrane is coated with specific binding molecules, specifically titanium-functionalized nanopolymer dendrimer, to enable the specific capture of a specific class of modified proteins, specifically phosphoproteins. B cell lymphoma cell lysate was spiked with either phosphorylated GST-cJun or unphosphorylated GST-cJun each at a 200:1 ratio. Sub-μL amounts of each mixture was spotted in dot-blot fashion onto a modified membrane. Immobilized cJun was detected using anti-GST primary antibody and fluorophore-functionalized anti-rabbit secondary antibody. In parallel, spotting, incubation and detection procedure was carried out with a control nitrocellulose membrane.

The comparative results at the same detection intensity are shown in FIG. 4. As the data reveals, the signal intensity from phosphorylated cJun is strong for both modified and control membranes, demonstrating a more complete, specific capture of the phosphorylated cJun onto the modified membrane. The detection limit is also similar for both, enabling detection of GST-cJun at the lowest spotted amount, 3.7 pg. In the case of the modified membrane, however, only the phosphorylated form of GST-cJun is detectable at the lowest spotted amount, 3.7 pg, indicating the specific capture of phosphorylated GST-cJun.

### Example #2

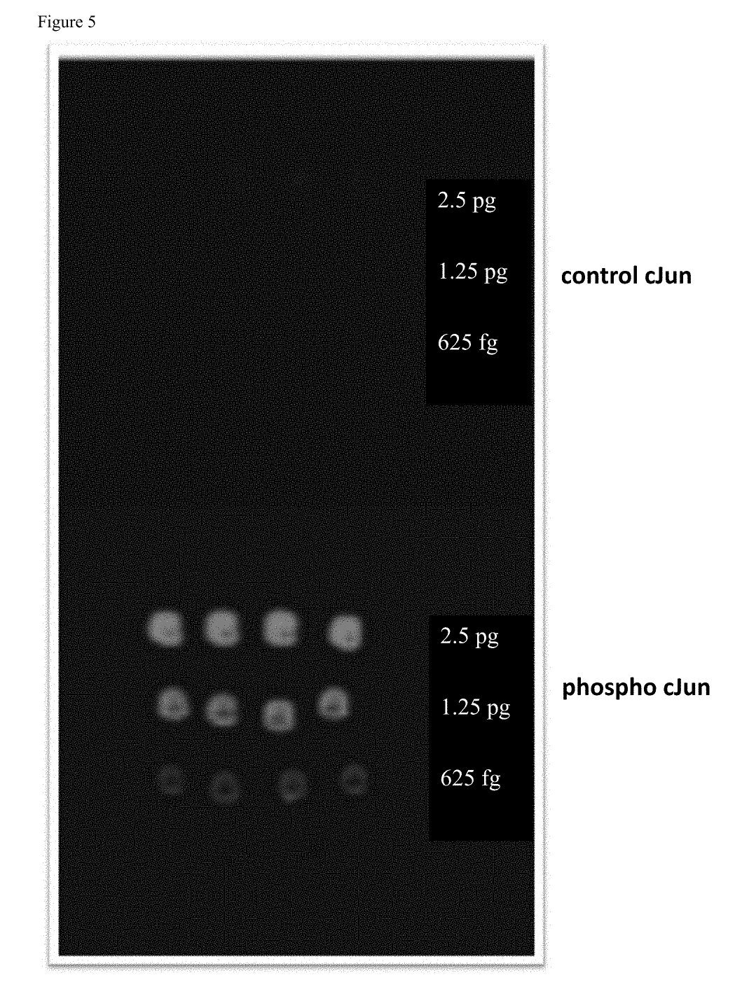

For Example #2, a simple dot-blot style spotting was used. A standard robotic spotting microarrayer is used in FIG. 5 to reduce the spotted sample volume to low-nL or sub-nL, and thus concentrates the signal in a smaller area and increases sensitivity.

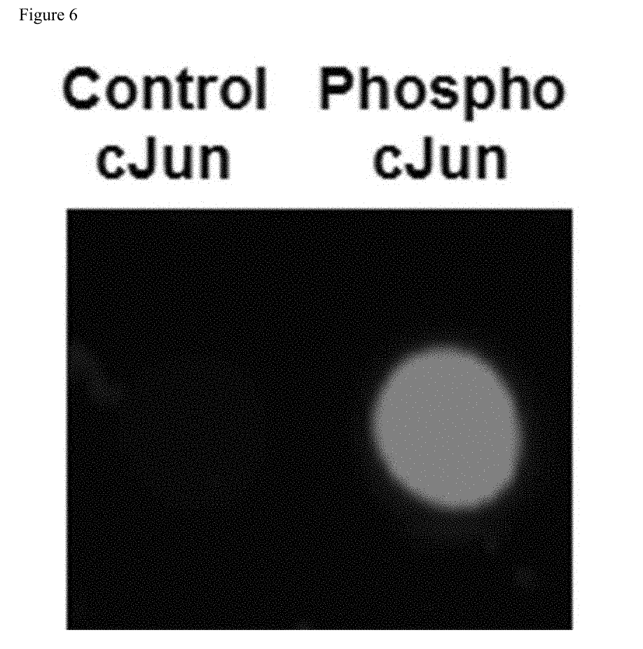

The binding capacity of the modified membrane is very high, likely due to nano-size of the specific binding molecules, which significantly increases the surface binding area of the reagent. Because of the high binding capacity and the ability to simplify the sample significantly by enriching only the modified proteins, a much higher amount of starting material is used. A typical starting sample limit for standard RPPAs is 1 μg/μL due to platform binding capacity. With the modified membrane, the sample limit can be increased, thus possibly providing improved detection of low-abundant modified proteins. As an example, phosphorylated and unphosphorylated forms of GST-cJun were spiked into complex undiluted plasma sample at a 1:10,000 ratio. The starting total protein concentration of each mixture was 50 μg/μL. Sub-μL amount of each sample was spotted on dot-blot fashion onto the modified membrane. Immobilized c-Jun was detected using anti-GST primary antibody and fluorophore-functionalized anti-rabbit secondary antibody. Plasma does not contain a high concentration of phosphorylated proteins. Capture of phospho-cJun was complete and detectable, as shown in FIG. 6. Control cJun did not produce a detectable signal.

A “systems approach” to study changes in protein modifications such as phosphorylation, a primary mode of cellular signaling, would enable a more in-depth analysis of drug targets and therapeutic efficacy. Currently, most commonly used drug screens detect only one or few of the related kinases, particularly for kinase inhibitors. However, due to inherent promiscuity of inhibitors, such as kinase inhibitors, a more extensive examination of signaling pathways is warranted for analysis of system perturbations and off-target effects—a common cause of drug failure.

### Example #3

The typical Glyco-RPPA (capture by PolyMAC-O—NH₂) protocol is to: (1) Oxidize a purified glycoprotein, such as Alpha-1-acid glycoprotein (AGP), with periodate by making an oxidation buffer composed of 100 mM sodium acetate adjusted to pH 5.5, preparing a protein sample that is 1 mg of protein per 1 mL of oxidation buffer, oxidizing the buffered sample by adding 10 mM periodate and allowing it to react in the dark for 30 minutes, then quenching the oxidation by adding 50 mM sodium sulfite and allowing to react in the dark for 15 minutes; (2) Denature the sample by boiling for 5 minutes in a denaturing buffer composed of 2% SDS and 2% 2-mercaptoethanol; (3) Serial dilute the samples using a dilution buffer composed of 1% SDS in phosphate buffered saline, and store in a freezer until ready for analysis. Following this procedure, the samples can be spotted and detected as described in the phospho-RPPA protocol.

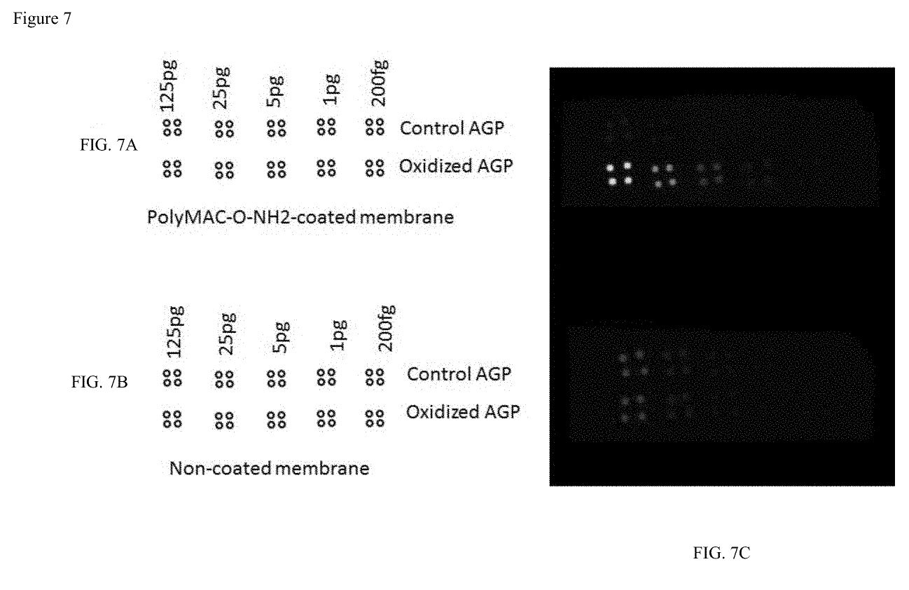

In one example, an O—NH₂-functionalized nanopolymer (PolyMAC variation) is immobilized to a nitrocellulose membrane to enable the specific capture of glycoproteins. A 5-fold serial dilution of oxidized or control (i.e., un-oxidized) AGP protein were spotted onto the membranes. The low-nL amount of this sample was spotted using an array pin onto the modified membrane, and the AGP signal detected using the primary antibody and fluorophore-functionalized anti-rabbit secondary antibody. In parallel, the identical spotting, incubation and detection procedure was carried out with unmodified nitrocellulose membrane. The comparative results at the same detection intensity are shown in FIG. 7. FIG. 7C shows a comparison between control and modified membrane for capture of the proteome/glycoproteome from cell culture. As the data reveals, the signal intensity from oxidized AGP is very strong and specific, with very little un-oxidized AGP detectable on the modified membrane.

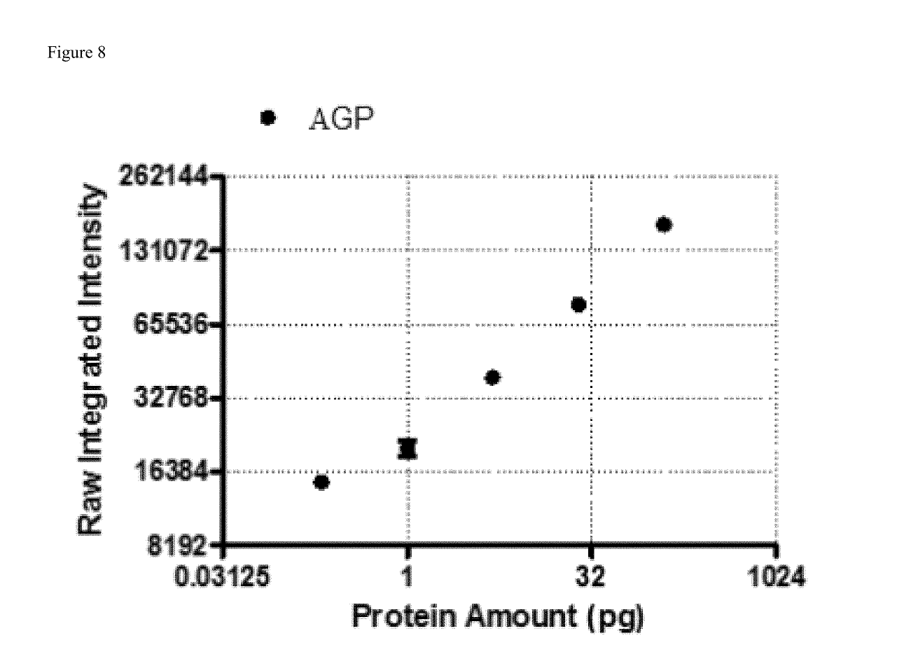

The signal from modified membrane demonstrated 25-fold intensity increase compared to control membrane due to better protein orientation on PolyMAC-O—NH₂ coated membrane. The selectivity is improved with the 125-fold signal increase due to periodate oxidation and formation of aldehydes. The modified membrane enables very selective capture of glycoproteins with greater than 99% specificity. The sensitivity is 1 pg of AGP, which is equivalent to 0.025 fmol for the 40 kDa protein. The signal is linear above 1 pg of AGP, as illustrated in FIG. 8.

### Example #4

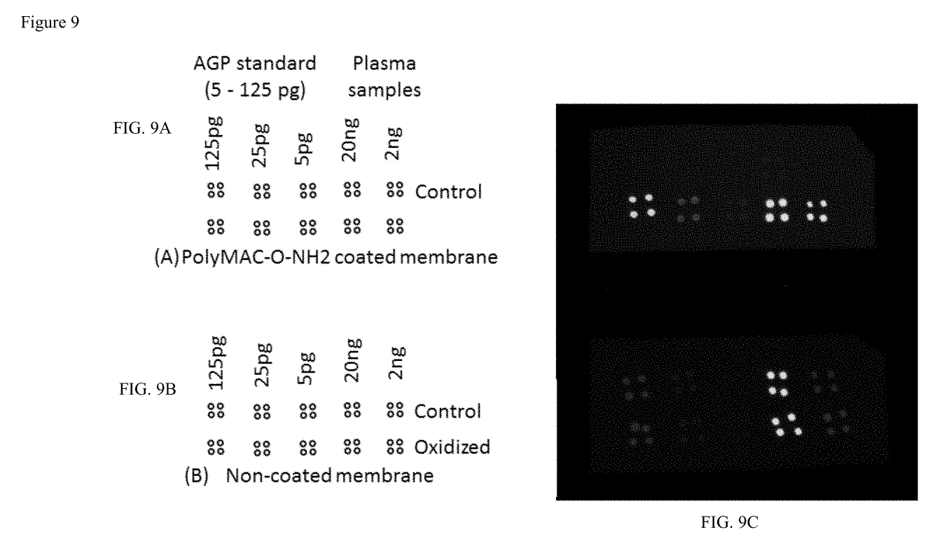

In another example, as illustrated in FIG. 9C, endogenous AGP protein from plasma is detected directly (right side of array in FIG. 9C based on legends FIGS. 9A and 9B) and compared to control AGP standard (left side of array in FIG. 9C based on legends FIGS. 9A and 9B). As in previous results, oxidized AGP can be detected from directly spotted plasma sample, while the un-oxidized version had hardly any signal on the PolyMAC-O—NH₂ membrane. Similarly, the signal from modified membrane was much stronger than from control membrane due to improved protein orientation.

What is claimed is:

1. A composition for binding a modified protein to a solid support comprising:
a pre-assembled binding moiety with an active binding molecule,
wherein the binding moiety includes at least one component selected from the group consisting of a polymer, nanopolymer, a dendrimer molecule, and a spacer link,
wherein the binding moiety is selected from the group consisting of metal ions, hydrazide, hydroxylamine, aldehyde, carbonyl groups, azide, alkyne, thiol, equivalents and combinations thereof,
wherein the binding moiety is coated onto to a solid support,
wherein the solid phase is a membrane, glass or plastic slide, platform of a reverse phase protein array, or any other type of protein array.

2. The composition of claim 1 wherein the solid phase is a nitrocellulose membrane.

3. The composition of claim 1 wherein the solid phase is a part of an immunoassay.

4. The composition of claim 1 wherein the detection moiety is part of a reverse phase or other types of protein or peptide array.

5. The composition of claim 1 wherein the binding moiety includes a dendrimer,
and the dendrimer includes hydrazide and hydroxylamine,
wherein said dendrimer binds with aldehyde for capture of glycosylation modifications.

6. A method for the detection of modified biological analyte in a sample comprising the steps of:
capturing a modified protein by contacting a sample to a solid support coated with the binding moiety, which includes an active binding molecule and at least one component selected from the group consisting of a polymer, nanopolymer, a dendrimer molecule, and a spacer link, such that the binding moiety selectively binds to a modification of a protein of the sample,
removing at least a portion of any unbound sample, and
analyzing the modified protein using a detection moiety selected from the group consisting of antibodies, aptamers, affirmers, lectins, equivalents and combinations thereof.

7. The method of claim 6 wherein the solid phase is a membrane, glass or plastic slide, platform of a reverse phase protein array, or any other type of protein array.

8. The method of claim 6 wherein the solid phase is a part of an immunoassay.

9. The method of claim 6 wherein the detection moiety is part of a reverse phase or other types of protein or peptide array.

10. The method of claim 6 wherein multiple samples are ran in parallel.

11. A method for the simultaneous capture and analysis of total protein and modified protein in a sample comprising the steps of:
retaining a first portion of the solid support for total protein binding,
and onto at least a second portion coating a binding moiety, which includes an active binding molecule and at least one component selected from the group consisting of a polymer, nanopolymer, a dendrimer
contacting the first portion and at least one additional portion of the support with a sample, such that the first portion binds total protein in the sample, and a binding moiety selectively binds modified protein in the sample,
removing at least a portion of any unbound sample, and
analyzing the bound protein using a detection moiety selected from the group consisting of antibodies, aptamers, affirmers, lectins, equivalents and combinations thereof.

12. The method of claim 11 where samples are ran in parallel.

13. The method of claim 11 where multiple and different binding moieties are coated in different portions of the solid support.

14. (canceled)
